(19) United States
(12) Patent Application Publication
(10) Pub. No.: US 2009/0054844 A1
(43) Pub. Date: Feb. 26, 2009

(54) MEDICAL GARMENT WRAP

(75) Inventors: Karen Alyea, Carrollton, TX (US); Andrea E. Simons, Carrollton, TX (US)

Correspondence Address: Glast, Phillips & Murray, P.C, 13355 Noel Road, Suite 2200, Dallas, TX, 75240, US

(73) Assignee: SIYA, INC., Carrollton, TX (US)

(21) Appl. No.: 12/195,820

(22) Filed: Aug. 21, 2008

Related U.S. Application Data
(60) Provisional application No. 60/957,746, filed on Aug. 24, 2007.

(51) Int. Cl. A61M 25/02; A61M 5/00

(52) U.S. Cl. 604/179; 604/174

(57) ABSTRACT

A medical garment wrap for safely holding lines or tubing implanted in a human or animal patient for medical purposes. The garment has a wrap portion for passing around a patient's body. The wrap portion has a pouch for holding multiple incoming lines. The pouch is closed by fasteners to hold the lines. The hub of the line into the patient's body is received and held in a hub holder. Typically several lines divide from the hub holder into ports, and the ports are held by one or more holders for lines and clamps. The pouch, the hub holder and the line holders are attached to the wrap portion. One end of the wrap has fingers with hook-and-loop fasteners that define an opening for passage of the line from the hub holder into the patient's body, through the wrap portion when the fingers are fastened to the opposite end of the wrap portion.

### CLAIM FOR PRIORITY

This application claims the priority of U.S. Provisional Patent Application Ser. No. 60/957,746, titled “Medical Garment Wrap,” filed Aug. 24, 2007, which application is incorporated by reference into the present application.

### BACKGROUND

1. Technical Field

This disclosure relates to garments or wraps intended to support and hold surgical lines or tubing implanted in a human or animal body. Such lines can include any intravenous line, catheter, feeding tube or drainage tube.

2. Background Art

In patients who have surgically implanted medical tubing or lines (the terms are used interchangeably in this disclosure) in their upper chest or upper abdominal area, the medical tubing is usually left to hang straight down off a patient's chest area. This allows easy access for the patient to pull it or tamper with it, so that the tubing may be completely removed from the patient's body. The result can be life-threatening due to hemorrhage and increased risk of infection. This in turn leads to emergency fixes by a team of medical personnel, and can ultimately lead to surgery. Allowed to hang freely, the tubing also becomes kinked and obstructed. This obstruction can interrupt the flow of treatment medications or solutions being administered via an external pump device. The external pump device emits an alarm requiring medical caregivers to solve the obstructions. Not only does this interrupt treatment, but also requires time of medical caregivers. The weight of the free-hanging tubing adds tension stress to the insertion site that is sutured (stitched) into place. This creates increased risk for skin irritation and discomfort at the insertion site. Multiple caretakers changing dressings frequently can lead to increased risk of infection and increased risk of mis-identification of differentiated ports. Ports and safety clamps on the tubing cause pressure to the skin and underlying tissues if not padded.

Past means of avoiding this situation, led to more tape on the patient, or a netted gauze material shaped into a “tube top.” Neither approach worked, as tape did not provide freedom of movement, and is irritating to the skin. Also, netted gauze shaped into a “tube top” had to be fashioned daily; the tube top did not stay in place on the patient, and the medical tubing had to be pulled through the very tiny net hole. The netted gauze still had to use tape to stay in place. Finally, access to the tubing was restricted requiring more time for care.

What is needed is a way to overcome the foregoing difficulties and maintain patency of tubing by preventing kinking that blocks the flow of medications or solutions to the patient. At the same time, it is highly desirable to prevent pressure of the tubing, ports and clamps on the tissues of the skin. This pressure is uncomfortable and carries the risk of breakdown injury to the skin and underlying tissues.

### DRAWINGS

FIG. 1 is a perspective view of the preferred embodiment of the medical garment wrap.

FIG. 2 is a cross-section of the wrap of the preferred embodiment.

FIGS. 3 and 4 represent the preferred embodiment of the wrap positioned on a patient, back view and front view, respectively.

FIGS. 5 and 6 show the preferred embodiment of the wrap open and laid flat, front and back view, respectively.

FIG. 7 is a detailed view of the portion of the wrap of the preferred embodiment, showing a holder for the hub of the incoming tubing and individual port holders.

### DESCRIPTION

FIG. 1 shows the garment (100) of the preferred embodiment. The wrap (110) portion of the garment (100) passes around the patient's body and is there supported by over-the-shoulder straps (160). The wrap (110) has attached a pouch (120) for holding multiple ports from the incoming tubing. Hook-and-loop fasteners (150 and 155) preferably close the pouch. Typical incoming tubing has a hub (not shown) that is received and held in a hub holder (140), again preferably by hook-and-loop fasteners. Typically several lines divide from the hub holder into ports (not shown), and these are held by one or more holders (130) for lines and clamps on the lines, also preferably closed by hook-and-loop fasteners. It is also preferable that the line holders (130) be color-coded to correspond to color-coded ports in the tubing.

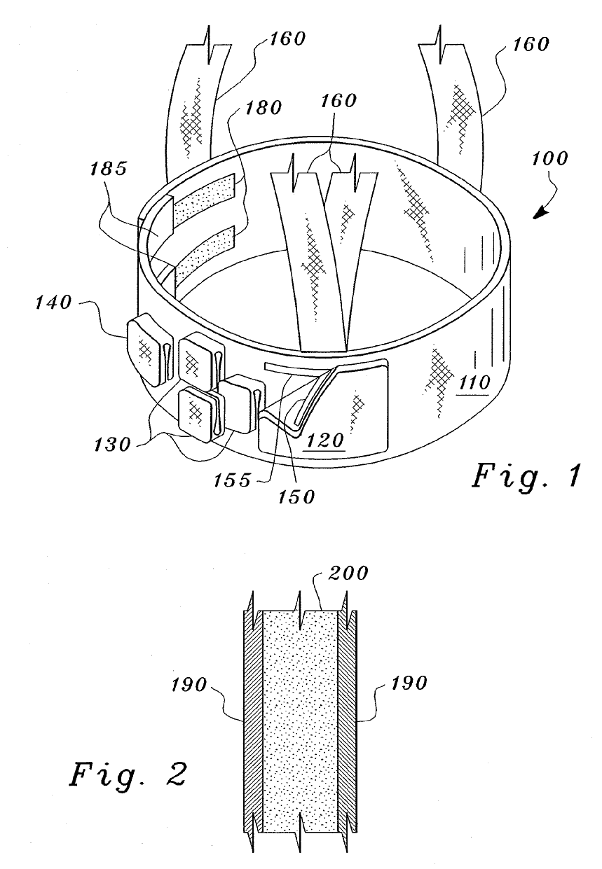

FIG. 2 shows a cross-section of the wrap (110), preferably comprising outer layers (190) of a washable soft fabric covering internal padding (200). The fabric and padding could be of cotton. The wrap (110) could also be constructed of synthetic material, such as rayon, nylon, polyester, or a polyethylene sheeting such as TYVEK (a trademark of E. I. Du Pont De Nemours Corp.), or equivalent materials.

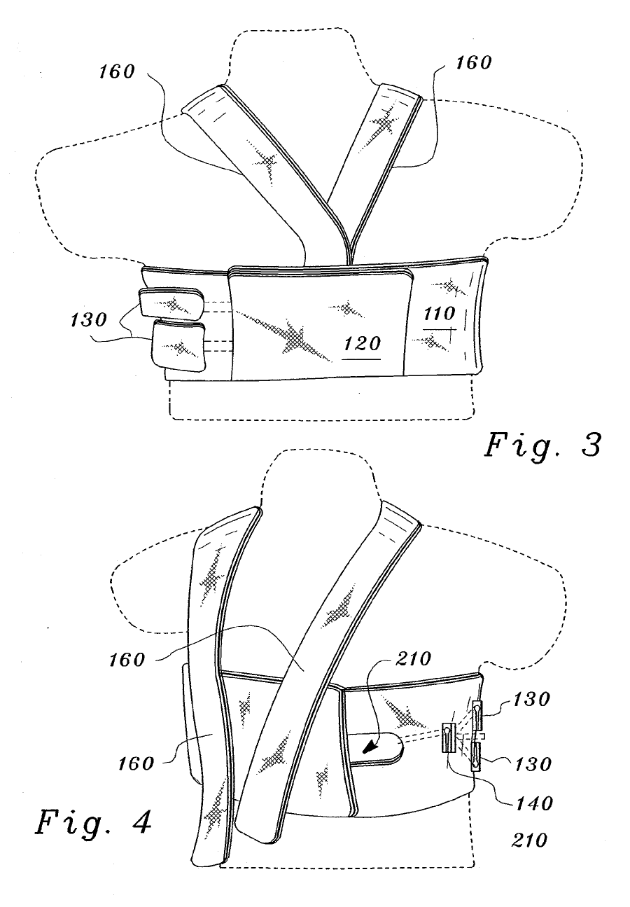

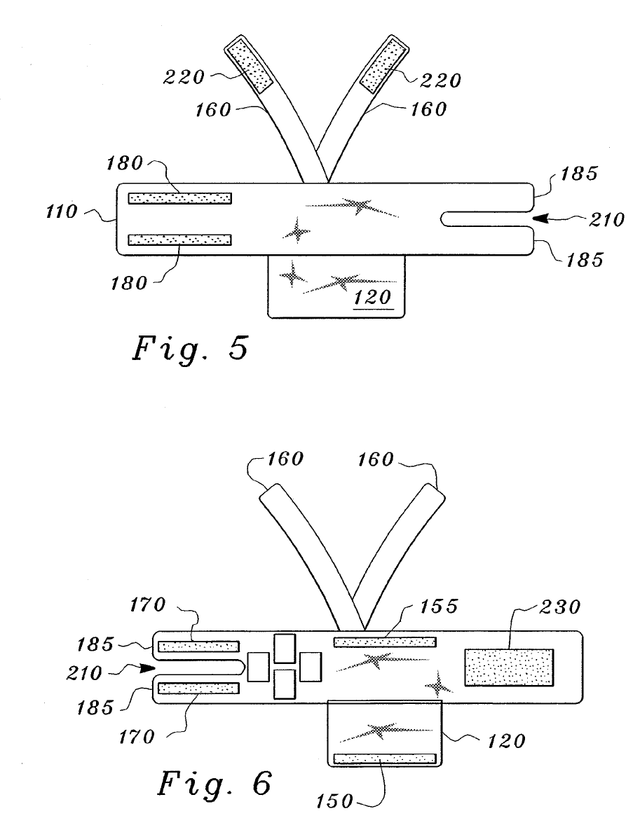

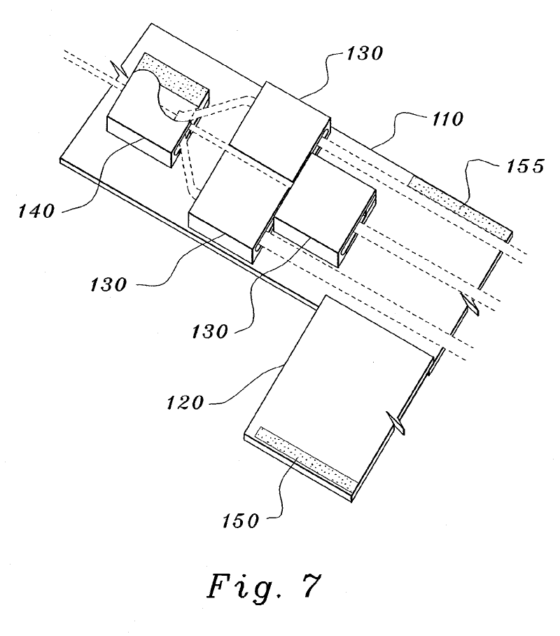

FIGS. 3 and 4 represent the preferred embodiment of the wrap positioned on a patient, back view and front view, respectively. FIGS. 5 and 6 show the preferred embodiment of the wrap open and laid flat, front and back view, respectively. Referring to these figures, the wrap (110) of the preferred embodiment is constructed as described in the following paragraphs. The following table of reference numerals corresponds to those in the figures:

| Reference | Item |
|---|---|
| 100 | entire garment |
| 110 | wrap portion of garment |
| 120 | pouch for holding ports from line |
| 130 | holders for separate lines and clamps |
| 140 | hub holder |
| 150 | hook-and-loop - loop side |
| 155 | hook-and-loop - hook side |
| 160 | straps |
| 170 | hook-and-loop on fingers 185 engaging 180 - hook |
| | side |
| 180 | hook-and-loop - loop side |
| 185 | fingers in wrap |
| 190 | outer fabric of wrap |
| 200 | padding inside wrap |
| 210 | opening defined by fingers 185 |
| 220 | hook-and-loop on straps 160 - hook side |
| 230 | hook-and-loop for attaching straps 160 - loop side |

The wrap has fingers (185) on one end, where the fingers (185) define an opening (210) for passage of lines to the patient. The fingers have hook-and-loop fasteners (170), which hook-and-loop fasteners (170) engage corresponding hook-and-loop fasteners (180) on the opposite side of wrap (110). In the preferred embodiment, any hook-and-loop fasteners that could contact the skin of the patient are arranged so that the soft loop side of the hook-and-loop fasteners is presented. The shoulder straps (160) connected to the wrap (110) have hook-and-loop fasteners (220) that engage an hook-and-loop fastener area (230) on the wrap, as shown in FIG. 6, thus holding the garment (100) securely to the patient. The shoulder straps (160) and the wrap (110) may be manufactured in different sizes to accommodate a variety of patient sizes and needs. Two straps (160) are preferred for stability of the wrap, but one strap (160) could suffice.

The pouch (120) for holding ports from an incoming line also is preferably closed by hook-and-loop fasteners (150, 155).

As shown in the figures, one side of the wrap (110) fits comfortably over opening (210) and attaches with hook-and-loop fasteners (170, 180) to the other end of the wrap (110). Medical tubing, especially surgically-implanted tubing, can then be wrapped around to the back of the patient's body, passing through the hub-holding pouch (140). Then, the tubing and clamps can be threaded through and fastened into the preferably color-coded line holders (130). The line holders (130), being padded, cushion the clamps from the patient and reduce the risk a clamp could become unclamped and allow air into a line. Ports connected to the lines are supported by the pouch (120), thus also cushioning the ports from the patient. As shown in FIG. 3, the shoulder straps (160) start approximately in the center of the wrap (110) in the rear, and come over the front of the patient's shoulders, and are held in place by hook-and-loop fasteners (220, 230).

It should be noted that other means for fastening the garment (100) to a patient, and for fastening tubing and ports to the garment (100) could be used, such as metal or plastic hooks engaging clasps or loops, zippers, or buttons.

We claim:

1. A medical garment wrap, comprising:
a wrap portion, the wrap portion capable of passing around a patient's body,
the wrap portion having an interior side facing the patient's body and an exterior side facing away from the patient's body;
a first end portion and a second end portion;
a middle portion disposed between the first end portion and the second end portion;
at least one strap, the least one strap connected to a first side of the wrap portion and a second side of the wrap portion,
the first side of the wrap portion and the second side of the wrap portion separated by the patient's body when the medical garment wrap is worn by the patient;
the least one strap capable of being supported by the patient's body when worn by the patient;
a pouch on the exterior side on the middle portion;
at least one fastener attached to the pouch corresponding to at least one fastener attached to the wrap portion;
the fasteners disposed to allow the pouch to fasten in a closed position;
the first end portion comprising at least two fingers, at least one fastener attached to each finger;
at least one fastener attached to the second end portion, such that the fasteners attached to the fingers correspond to and attach to the at least one fastener attached to the second end portion; and,
an opening defined by the fingers and the second end portion when the fingers are connected by the fasteners to the second end portion, the opening being capable of allowing medical tubing to pass therethrough.

2. The medical garment wrap of claim 1, further comprising:
outer layers of a washable soft fabric covering internal padding, wherein the fabric and the padding are each made from at least one of cotton, rayon, nylon, polyester or polyethylene sheeting.

3. The medical garment wrap of claim 1,
wherein the fasteners are hook-and-loop fasteners.

4. The medical garment wrap of claim 1,
wherein at least one connection between the at least one strap and the wrap portion is comprised of at least one fastener on the at least one strap corresponding and attaching to at least one fastener on the wrap portion.

5. The medical garment of claim 1, further comprising:
a hub holder on the exterior side on the middle portion disposed closer to the first end portion than the pouch;
at least one fastener attached to the hub holder corresponding and attaching to at least one fastener attached to the wrap portion;
the fasteners being disposed to allow the hub holder to fasten in a closed position to hold a tubing hub.

6. The medical garment wrap of claim 5, further comprising:
a plurality of line holders on the exterior side on the middle portion disposed between the hub holder and the pouch;
the line holders being padded;
at least one fastener attached to each line holder pouch corresponding and attaching to at least one fastener attached to the wrap portion;
the fasteners being disposed to allow each line holder pouch to fasten in a closed position.

7. The medical garment wrap of claim 6,
wherein the line holders are color coded.

8. A medical garment wrap, comprising:
a wrap portion, the wrap portion capable of passing around a patient's body, the wrap portion having an interior side facing the patient's body and an exterior side facing away from the patient's body;
a first end portion and a second end portion;
a middle portion disposed between the first end portion and the second end portion;
least one strap, the least one strap connected to a first side of the wrap portion and a second side of the wrap portion,
the first side of the wrap portion and the second side of the wrap portion separated by the patient's body when the medical garment wrap is worn by the patient;
the least one strap capable of being supported by the patient's body when worn by the patient;
a pouch on the exterior side on the middle portion;
at least one fastener attached to the pouch corresponding to at least one fastener attached to the wrap portion;
the fasteners being disposed to allow the pouch to fasten in a closed position;
at least one fastener attached to the first end portion; and,
at least one fastener attached to the second end portion, such that the at least one fastener attached to the first end portion correspond to and attach to the at least one fastener attached to the second end portion.

9. The medical garment wrap of claim 8, further comprising:
the first end portion being comprised of at least two fingers, at least one fastener attached to each finger;
an opening defined by the fingers and the second end portion when the fingers are connected by the fasteners to the second end portion, the opening being capable of allowing medical tubing to pass therethrough.

10. The medical garment wrap of claim 8, further comprising:
outer layers of a washable soft fabric covering internal padding,
wherein the fabric and the padding are each made from at least one of cotton,
rayon, nylon, polyester or polyethylene sheeting.

11. The medical garment wrap of claim 8,
wherein the fasteners are hook-and-loop fasteners.

12. The medical garment wrap of claim 8,
wherein at least one connection between the least one strap and the wrap portion is comprised of at least one fastener on the least one strap corresponding and attaching to at least one fastener on the wrap portion.

13. The medical garment of claim 8, further comprising:
a hub holder on the exterior side on the middle portion disposed closer to the first end portion than the pouch;
at least one fastener attached to the hub holder corresponding to and attaching to at least one fastener attached to the wrap portion,
the fasteners being disposed to allow the hub holder to fasten in a closed to hold a tubing hub.

14. The medical garment wrap of claim 8, further comprising:
a plurality of line holders on the exterior side on the middle portion disposed between the hub holder and the pouch,
the line holders being padded;
at least one fastener attached to each line holder pouch corresponding to and attaching to at least one fastener attached to the wrap portion;
the fasteners being disposed to allow each line holder pouch to fasten in a closed position.

15. The medical garment wrap of claim 14,
wherein the line holders are color coded.
